(19) United States
(12) Patent Application Publication
(10) Pub. No.: US 2023/0310199 A1
(43) Pub. Date: Oct. 5, 2023

(54) WRAP SYSTEM FOR IMMOBILIZING A PERSON

(71) Applicant: Melior Motus Inc., Halifax (CA)

(72) Inventors: Stephen Douglas BEED, Halifax (CA); Paul LAPSTUN, Collaroy Beach (AU)

(21) Appl. No.: 18/295,471

(22) Filed: Apr. 4, 2023

Related U.S. Application Data
(60) Provisional application No. 63/362,493, filed on Apr. 5, 2022.

(51) Int. Cl. A61F 5/37

(52) U.S. Cl. CPC A61F 5/3776

(57) ABSTRACT

A wrap system for immobilizing a spine of a person, including a wrap dimensioned to be snugly securable around a person and extending from at least the upper torso to at least below knees of the person to restrict independent movement of limbs of the person. The wrap includes a support structure including at least one fluid chamber positionable along a posterior side of the person when the wrap is secured around the person. The fluid chamber is reopenably sealable to enclose a fluid therein, the support structure immobilizing relative movement of a spine of the person.

### CROSS-REFERENCE TO RELATED APPLICATIONS

This application claims the benefit of U.S. Provisional Application No. 63/362,493, filed Apr. 5, 2022, the contents of which are incorporated herein by reference in their entirety.

### FIELD

The specification relates generally to transporting a person and in particular to a wrap system for immobilizing a person.

### BACKGROUND OF THE DISCLOSURE

The evacuation and transportation of a critically ill patient from a variety of locations to a treatment and assessment center can be very challenging where it is desired to protect the patient from major injuries or exacerbation of such injuries like a C-spine fracture, fractured pelvis, long bone fractures, etc. In addition, the patient may be agitated, delirious, in pain, etc. Resuscitation of the patient en route may be required.

Currently, medics may “scoop and run”, putting the patient on a canvas gurney and hoping that the patient's injuries are not exacerbated in the process of transportation. Alternatively, the patient may be put on a stretcher and airlifted via helicopter.

### SUMMARY OF THE DISCLOSURE

In a first aspect of the present disclosure, there is provided a wrap system having a fluid-filled support structure, comprising a wrap dimensioned to be snugly securable around a person and extending from at least the upper torso to at least below knees of the person to restrict independent movement of limbs of the person, the wrap including a support structure including at least one fluid chamber positionable along a posterior side of the person when the wrap is secured around the person, the fluid chamber being reopenably sealable to enclose a fluid therein, the support structure immobilizing relative movement of a spine of the person.

In other embodiments, the support structure can be separate from the wrap and securable to the wrap generally along the wrap adjacent to the posterior side of the person when the wrap is secured thereon.

The wrap can include a head support structure having an attachment structure for attaching the head support structure to the wrap when positioned around a person, the attachment structure restricting movement of the head support structure relative to the wrap, the head support structure having a head restraint structure restricting movement of a head of the person.

The head restraint structure can include a strap extending across the head of the person to restrict movement of the head relative to the head support structure.

The head restraint structure can include lateral support members extending along lateral sides of the head of the person to restrict rotation of the head.

The head restraint structure can include a fluid chamber for cushioning the head of the person when positioned thereon.

The wrap can have an opening along at least one side thereof.

The wrap can include hip straps to further restrict movement of a pelvis of the person.

A chest port can be provided in the wrap to facilitate access to a chest of the person.

A vein port can be provided in the wrap to facilitate access to an antecubital fossa of the person.

The support structure can include at least a second fluid chamber that is positionable along the posterior side of the person when the wrap is secured around the person, the at least a second fluid chamber being reopenably sealable to enclose a fluid therein.

The wrap can have sleeves extending generally along a head-to-foot axis of the person for receiving stiffener members.

A transportation structure can be coupleable to the wrap to provide rigidity to the wrap.

The transportation structure can include two longitudinal sections extending along the wrap along a generally head-to-foot axis of the person when the wrap is secured thereon.

The transportation structure can include two lateral sections extending between the two longitudinal sections towards distal ends thereof.

Each longitudinal section can be formed from two longitudinal section ports that are securable to one another, and each lateral section is formed from two lateral section portions that are securable to one another.

Each longitudinal section portion can be coupled to a corresponding one of the lateral section portions.

Each longitudinal section portion can be pivotally coupled to a corresponding one of the lateral section portions.

A foot wheel structure can be coupleable to or form part of the wrap towards feet of the person when the wrap is secured thereon, comprising a wheel for facilitating rolling travel over a surface.

The foot wheel structure can include an inflatable boot structure that is dimensioned to be positioned over at least feet of the person.

A torso wheel structure can be coupleable to or form part of the wrap towards shoulders of the person when the wrap is secured thereon, and can include at least two wheels for facilitating rolling travel over a surface.

In accordance with a second aspect of the present disclosure, there is provided a wrap system for immobilizing a person, comprising a wrap dimensioned to be snugly securable around a person and extending from at least the upper torso to at least below knees of the person to restrict independent movement of limbs of the person, the wrap including a support structure including at least one fluid chamber positionable along a posterior side of the person when the wrap is secured around the person, the fluid chamber being reopenably sealable to enclose a fluid therein, the support structure immobilizing relative movement of a spine of the person.

In some or all exemplary embodiments of the second aspect, the support structure is dimensioned to provide a head support structure for restricting movement of a head of the person relative to the torso of the person, the head support structure having a head restraint structure restricting movement of the head of the person relative to the head support structure.

In some or all exemplary embodiments of the second aspect, the head restraint structure includes a strap extending across a forehead of the person.

In some or all exemplary embodiments of the second aspect, the head restraint structure includes lateral support members extending along lateral sides of the head of the person to restrict rotation of the head.

In some or all exemplary embodiments of the second aspect the head restraint structure includes a head-supporting fluid chamber for cushioning the head of the person when positioned thereon.

In some or all exemplary embodiments of the second aspect, the wrap system further includes a head support structure coupleable to or extending from the wrap for restricting movement of a head of the person relative to the torso of the person, the head support structure having a head restraint structure restricting movement of the head of the person relative to the head support structure.

In some or all exemplary embodiments of the second aspect, the head restraint structure includes a strap extending across a forehead of the person.

In some or all exemplary embodiments of the second aspect, the head restraint structure includes lateral support members extending along lateral sides of the head of the person to restrict rotation of the head.

In some or all exemplary embodiments of the second aspect, the head restraint structure includes a head-supporting fluid chamber for cushioning the head of the person when positioned thereon.

In some or all exemplary embodiments of the second aspect, the wrap includes hip straps to further restrict movement of a pelvis of the person.

In some or all exemplary embodiments of the second aspect, the wrap includes a chest port to facilitate access to a chest of the person when the wrap is secured around the person.

In some or all exemplary embodiments of the second aspect, the wrap includes a vein port to facilitate access to an antecubital fossa of the person when the wrap is secured around the person.

In some or all exemplary embodiments of the second aspect, the support structure includes at least a second fluid chamber that is positionable along the posterior side of the person when the wrap is secured around the person, the at least a second fluid chamber being reopenably sealable to enclose a fluid therein.

In some or all exemplary embodiments of the second aspect, the wrap includes at least two sleeves extending generally along a head-to-foot axis of the person for receiving stiffener members.

In some or all exemplary embodiments of the second aspect, the wrap system further includes a transportation structure that is coupleable to the wrap to provide rigidity to the wrap when the wrap secured around the person is being moved.

In some or all exemplary embodiments of the second aspect, the transportation structure includes two longitudinal sections extending along the wrap generally along a head-to-foot axis of the person when the wrap is secured around the person.

In some or all exemplary embodiments of the second aspect, the transportation structure includes two lateral sections extending between the two longitudinal sections towards distal ends thereof.

In some or all exemplary embodiments of the second aspect, each longitudinal section is formed from two longitudinal section portions that are securable to one another, and each lateral section is formed from two lateral section portions that are securable to one another.

In some or all exemplary embodiments of the second aspect, each longitudinal section portion is coupleable to a corresponding one of the lateral section portions.

In some or all exemplary embodiments of the second aspect, each longitudinal section portion is pivotally coupleable to a corresponding one of the lateral section portions.

In some or all exemplary embodiments of the second aspect, the wrap system further includes a foot wheel structure that is coupleable to or forms part of the wrap towards feet of the person when the wrap is secured around the person, the foot wheel structure including a wheel for facilitating rolling travel over a surface.

In some or all exemplary embodiments of the second aspect, the foot wheel structure includes an inflatable boot structure that is dimensioned to be positioned over at least the feet of the person.

In some or all exemplary embodiments of the second aspect, the wrap system further includes a torso wheel structure coupleable to or forming part of the wrap towards shoulders of the person when the wrap is secured around the person, the torso wheel structure including at least two wheels for facilitating rolling travel over a surface.

Other technical advantages may become readily apparent to one of ordinary skill in the art after review of the following figures and description.

### BRIEF DESCRIPTIONS OF THE DRAWINGS

For a better understanding of the embodiment(s) described herein and to show more clearly how the embodiment(s) may be carried into effect, reference will now be made, by way of example only, to the accompanying drawings in which:

FIG. 1 is a side elevation view of a wrap having a fluid-filled support structure in accordance with an embodiment;

FIG. 2 is a top plan view of the wrap of FIG. 1;

FIG. 3A is a top plan view of a transportation structure that is coupleable to the wrap of FIGS. 1 and 2 and having two telescoping longitudinal sections and two telescoping lateral sections;

FIG. 3B is a top plan view of the transportation structure of FIG. 3A being separated along each longitudinal section and each lateral section into four elements with the telescoping portions collapsed;

FIG. 3C is a top plan view of one of the four segments of FIG. 3B, wherein a lateral section portion is pivoted relative to a longitudinal section portion;

FIG. 3D is a side elevation view of the transportation structure of FIG. 3A coupled to the wrap of FIGS. 1 and 2;

FIG. 3E shows the four segments of FIG. 3C after pivoting of the lateral section portion relative to the longitudinal section portion so that they are generally parallel, with the telescoping portions extended;

FIG. 3F is a top plan view of the wrap of FIG. 1 secured to a person and coupled to the transportation structure of FIG. 3A;

FIG. 3G is a top plan view of the wrap and transportation structure of FIG. 3F, wherein the wrap is partially decoupled from the transportation structure;

FIG. 3H shows the transportation structure partially disassembled along the longitudinal portions with the wrap folded over;

FIG. 3I shows the transportation structure and folded wrap of FIG. 3H coupled to a backpack;

FIG. 3J is a is a top plan view of the transportation structure of FIG. 3A with a wheel structure secured thereto towards a foot end thereof;

FIG. 4A is a side elevation view of a wrap having a fluid-filled support structure in accordance with another embodiment, wherein a foot wheel structure is secured over feet of the person;

FIG. 4B is an end view of the wrap of FIG. 4A;

FIG. 5A is a top plan view of a transportation structure that is coupleable to the wraps of FIGS. 1 and 4 in accordance with another embodiment;

FIG. 5B is a top plan view of the transportation structure of FIG. 5A being separated along each longitudinal section and each lateral section into four elements with the telescoping portions collapsed;

FIG. 5C is a top plan view of one of the four segments of FIG. 5B, wherein a lateral section portion is being pivoted relative to a longitudinal section portion;

FIG. 5D is a side elevation view of the segment of FIG. 5B after pivoting of the lateral section portion relative to the longitudinal section portion;

FIG. 5E shows the transportation structure of FIG. 5A coupled to the wrap of FIGS. 1 and 2;

FIG. 6 is a side elevation view of a wrap having a separate fluid-filled support structure secured to it in accordance with a further embodiment; and

FIG. 7 shows a wrap system in accordance with yet another embodiment, wherein the wrap extends beneath the head and feet of the person.

Unless otherwise specifically noted, articles depicted in the drawings are not necessarily drawn to scale.

### DETAILED DESCRIPTION

For simplicity and clarity of illustration, where considered appropriate, reference numerals may be repeated among the Figures to indicate corresponding or analogous elements. In addition, numerous specific details are set forth in order to provide a thorough understanding of the embodiment or embodiments described herein. However, it will be understood by those of ordinary skill in the art that the embodiments described herein may be practiced without these specific details. In other instances, well-known methods, procedures and components have not been described in detail so as not to obscure the embodiments described herein. It should be understood at the outset that, although exemplary embodiments are illustrated in the figures and described below, the principles of the present disclosure may be implemented using any number of techniques, whether currently known or not. The present disclosure should in no way be limited to the exemplary implementations and techniques illustrated in the drawings and described below.

Various terms used throughout the present description may be read and understood as follows, unless the context indicates otherwise: “or” as used throughout is inclusive, as though written “and/or”; singular articles and pronouns as used throughout include their plural forms, and vice versa; similarly, gendered pronouns include their counterpart pronouns so that pronouns should not be understood as limiting anything described herein to use, implementation, performance, etc. by a single gender; “exemplary” should be understood as “illustrative” or “exemplifying” and not necessarily as “preferred” over other embodiments. Further definitions for terms may be set out herein; these may apply to prior and subsequent instances of those terms, as will be understood from a reading of the present description. It will also be noted that the use of the term “a” or “an” will be understood to denote “at least one” in all instances unless explicitly stated otherwise or unless it would be understood to be obvious that it must mean “one”.

Modifications, additions, or omissions may be made to the systems, apparatuses, and methods described herein without departing from the scope of the disclosure. For example, the components of the systems and apparatuses may be integrated or separated. Moreover, the operations of the systems and apparatuses disclosed herein may be performed by more, fewer, or other components and the methods described may include more, fewer, or other steps. Additionally, steps may be performed in any suitable order. As used in this document, “each” refers to each member of a set or each member of a subset of a set.

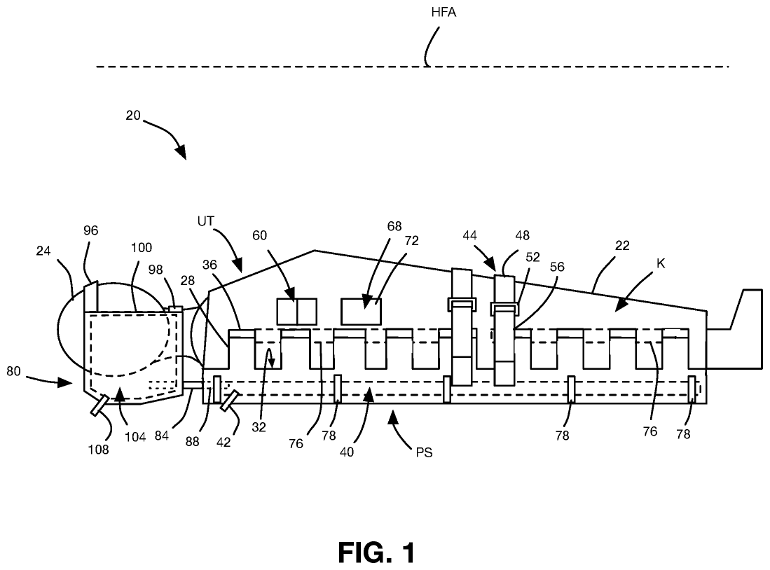

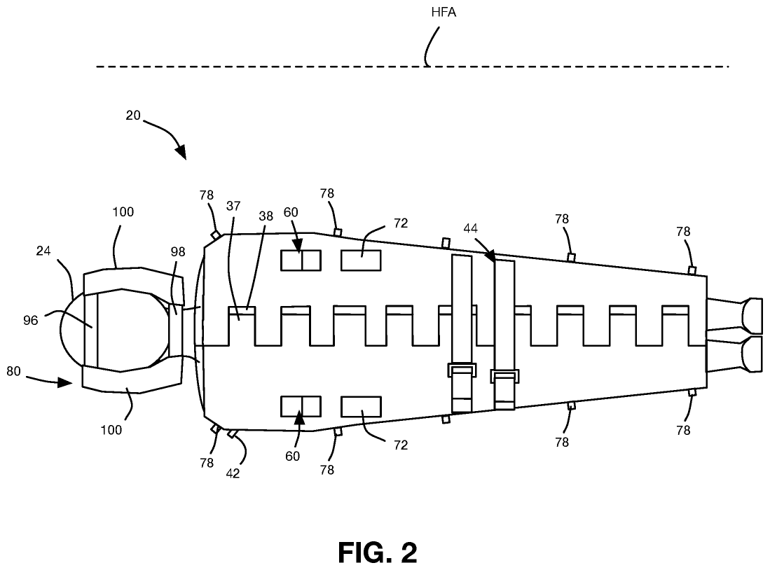

A wrap system 20 in accordance with an embodiment is shown in FIGS. 1 and 2. The wrap system 20 is used to transport a person who has been injured or who otherwise needs to be immobilized. In order to generally immobilize a person 24, a wrap 22 of the wrap system 20 is dimensioned to be snugly securable around the person 24 and extend from at least the upper torso UT to at least below knees K of the person. In this manner, the wrap 22 restricts independent movement of limbs (that is, the arms and legs) of the person 24. The wrap 22 can be principally made of any suitably strong material, such as ballistic nylon, cotton, polyester, etc.

In the embodiment illustrated in FIGS. 1 and 2, the wrap 22 is generally a unitary construction and can be opened along one lateral side. In other embodiments, however, the wrap 22 can be constructed from two or more sections that are securable to one another, and may be openable at more than one location.

In order to be snugly secured around the person 24, the wrap 22 includes a set of tabs 28 along one lateral edge 32 that have hook fabric patches on their undersides (not visible in this view) to engage (corresponding) loop fabric patches 36 along a second lateral edge (not visible in this view). As will be appreciated, the hook fabric patches and the loop fabric patches 36 are sufficiently sized to enable adjustment of the position of the tabs 28 relative to the loop fabric patches 36 to alter the circumference, and thus the snugness, of the wrap 22.

In addition, a set of tabs 37 along a joint on an anterior side of the person 24 have hook fabric patches on their undersides (not visible in this view) to engage (corresponding) loop fabric patches 38 along a second lateral edge (not visible in this view). As will be appreciated, the hook fabric patches and the loop fabric patches 38 are sufficiently sized to enable adjustment of the position of the tabs 37 relative to the loop fabric patches 38 to alter the circumference, and thus the snugness, of the wrap 22.

The wrap 22 has a support structure including at least one bladder 40 that is positioned along a posterior side PS of the person 24 when the wrap 22 is secured around the person 24. The bladder 40 extends substantially across the width of the person's posterior side PS but can be varied in width somewhat. Relative to a longitudinal head-to-foot axis HFA, the bladder 40 extends from at least the base of the neck to at least the pelvis. While the wrap 22 is shown having a single bladder, it will be appreciated that the wrap can have two or more bladders.

The bladder 40 is fully enclosed within the exterior ballistic nylon shell of the wrap 22, but can also be partially or fully externally positioned relative to the external ballistic nylon shell. Any suitably fluid-tight, flexible, and durable material can be used to construct the bladder 40. A valve 42 that is reopenably sealable enables fluid communication with the bladder 40 to fill the bladder 40 with a fluid, and to drain the fluid from the bladder 40. In the illustrated and described embodiment, the fluid is a gas such as air or carbon dioxide, but can be any other suitable fluid. The valve 42 can be designed to be either connected to a compressed air source, such as a carbon dioxide canister that may be held in the wrap 20, or orally blown. Alternatively, an external pump, such as a manual or electrical one, can be employed.

As the wrap 22 is principally made of flexible materials, when the bladder 40 is generally empty of air, the wrap 22 can be collapsed and folded or rolled to package the wrap 22 for carrying, such as in a backpack. When the bladder 40 is filled with air to a sufficient pressure, the bladder 40 becomes rigid. As a result, when the person 24 is snugly secured within the wrap 22 and the bladder 40 is filled, the support structure serves as a rigid backboard, and the spine of the person 24 is generally restricted from relative movement.

The support structure can be generally planar or can be contoured as desired to best support the person 24.

The wrap 22 has a set of wide hip straps 44 in the region of the pelvis of the person when secured in the wrap 22. The hip straps 44 include a first strap portion 48 having a bracket 52 and a second strap portion 56 that is passed through the bracket 52 and then folded back and adhered onto itself via hook-and-loop fabric or the like. This allows better tension on the hip straps 44 and thus snugness in the fit of the wrap 22 in the pelvic region to provide external stabilization of the pelvis. The hip straps 44 have a sufficiently high tensile strength to further restrict movement of a pelvis of the person 24, as this can be a particularly sensitive region that may be prone to injury. A tension indicator can be built into the hip straps 44, such as color coding, etc., to indicate the length (and thus a relative level of tension) of the hip straps 44 and thus the compression/immobilization provided.

Two chest ports 60 are provided in an anterior region of the wrap 22 to facilitate access to a chest of the person 24. In particular, access is provided by the chest ports 60 to the fifth intercostal space in the anterior axillary line on either side of the person 24. A set of straps 64 are securable to one another via hook-and-loop fabric patches to open and close the chest port 60. A chest tube can be inserted through the chest port 60 when the straps 64 are separated.

A pair of antecubital fossa ports 68 are provided in the wrap 22 to facilitate access to an antecubital fossa of the person 24. Access to the antecubital fossa (where large bore IVs are deployed) of either arm of the person 24 during transportation can be desirable. The antecubital fossa ports 68 can be opened and closed via a flap 72 that can be releasably closed in any suitable manner, such as hook-and-loop fabric patches.

In order to enhance the stiffness of the wrap, stiffener receptacles in the form of sleeves 76 are provided towards the lateral sides of the wrap 22 for receiving stiffener members (not shown), such as metal bars. The stiffener members extend generally along the head-to-foot axis HFA. These stiffener receptacles can be employed along the body of the person 24, but particularly along the legs. Stiffener members positioned in sleeves 76 along the legs can act as long bone splints for femur or tibial fractures.

A set of transport connectors in the form of nylon loops 78 are stitched onto the wrap 22 at positions along lateral sides of the wrap 22. The nylon loops 78 are reopenably closable via hook-and-loop fabric patches or the like.

At an assessment centre, it can be desirable to have a patient on a rigid board when examining their anterior side (chest, abdomen, etc.). In order to examine the front of a patient when supine, the straps 44 and the tabs 37 or tabs 28 can be opened to open up the wrap 22 to expose the front side of the patient. In order to examine the back of a patient, the patient can be put in a prone position after opening up the straps 44, and the tabs 28 opened to pull back the inflated backboard.

A head support 80 to support the head of the person 24 has an attachment structure for attaching the head support 80 to the wrap 22. The attachment structure restricts movement of the head support 80 relative to the wrap 22. In this embodiment, the attachment structure includes a rigid member in the form of a metal plate 84 extending towards the wrap 22. While the rigid member is made of metal in this particular embodiment, any other material or construction that is suitably resistant to bending and torqueing forces can be employed. The metal plate 84 is received within a sleeve 88 of the wrap 22. In one configuration, about three inches of the metal plate 84 is received within the sleeve 88. The snug fit and the shape of the metal plate 88 within the sleeve 92 restrict movement of the head support 80 relative to the wrap 22. Similarly, the metal plate 84 may be received within a sleeve of the head support 80 facilitating its detachment from the head support 80. The sleeve 88 of the wrap 22 and the sleeve of the head support 80 can include wings or the like to resist rotation of the wrap 22 and/or the head support 80 relative to the metal plate 84.

The head support 80 has a head restraint structure restricting movement of the head of the person 24. It has an exterior shell constructed of a material such as ballistic nylon or the like. In the illustrated embodiment, the head restraint structure includes a strap 96 extending across the head of the person 24 and a chinstrap 98 extending under the chin of the person 24 to restrict movement of the head relative to the head support 80. Alternatively or additionally, a neck strap can be employed to immobilize the head.

The head restraint structure can include lateral support members 100 extending along lateral sides of the head of the person 24 to restrict rotation of the head.

The neck and the face of the person 24 may be left exposed to facilitate treatment of the person 24 while secured in the wrap 22 and the head support 80, such as the performance of a surgical airway procedure.

A bladder 104 is positioned within the exterior shell of the head support 80. The bladder 104 extends across a posterior side of the head and around the lateral sides of the head in the lateral support members 100. It is fully enclosed within the exterior ballistic nylon shell of the head support 80, but can also be partially or fully externally positioned relative to the external ballistic nylon shell. Any suitably fluid-tight, flexible, and durable material can be used to construct the bladder 104. A valve 108 that is reopenably sealable enables fluid communication with the bladder 104 to fill the bladder 104 with a fluid, and to drain the fluid from the bladder 104. In the illustrated and described embodiment, the fluid is air, but can be any other suitable fluid. While the head support 80 is shown having a single bladder, it will be appreciated that the head support can have two or more bladders.

As the head support 80 is principally made of flexible materials, apart from the metal plate 88, when the bladder 104 is generally empty of air, the head support 80 can be collapsed and folded or rolled to package the wrap 22 for carrying, such as in a backpack. When the bladder 104 is filled with air to a sufficient pressure, the bladder 104 becomes rigid. As a result, when the head of the person 24 is positioned within the head support 80 and the bladder 104 is filled, the head support 80 restricts movement of the head relative to the body of the person 24. Thus, using the wrap system 20, the cervical, thoracic, and lumbar spine are kept rigid during transport.

As will be appreciated, the bladders 40, 104 within the wrap 22 and the head support 80 provide cushioning to reduce the impact of bumping with objects in an environment.

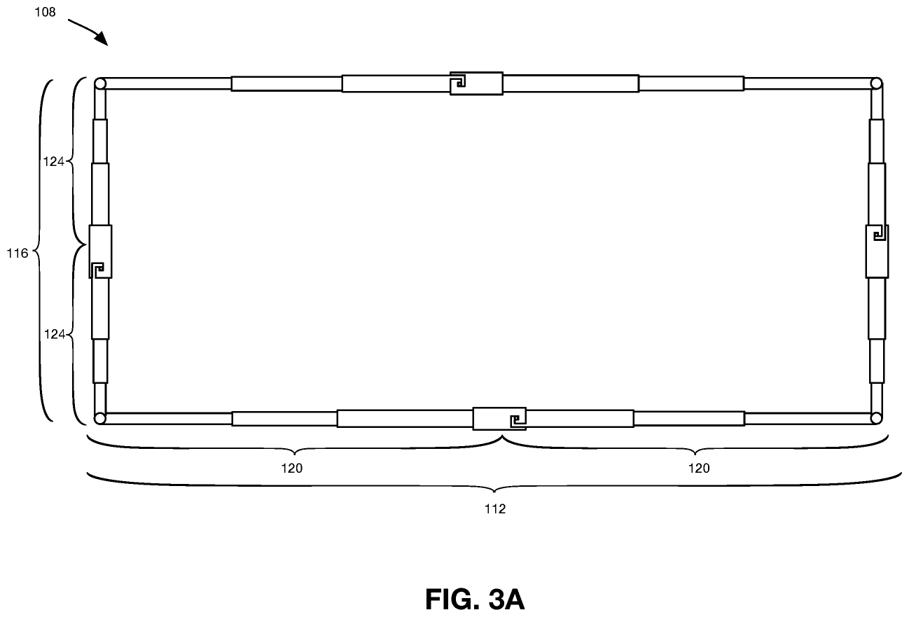

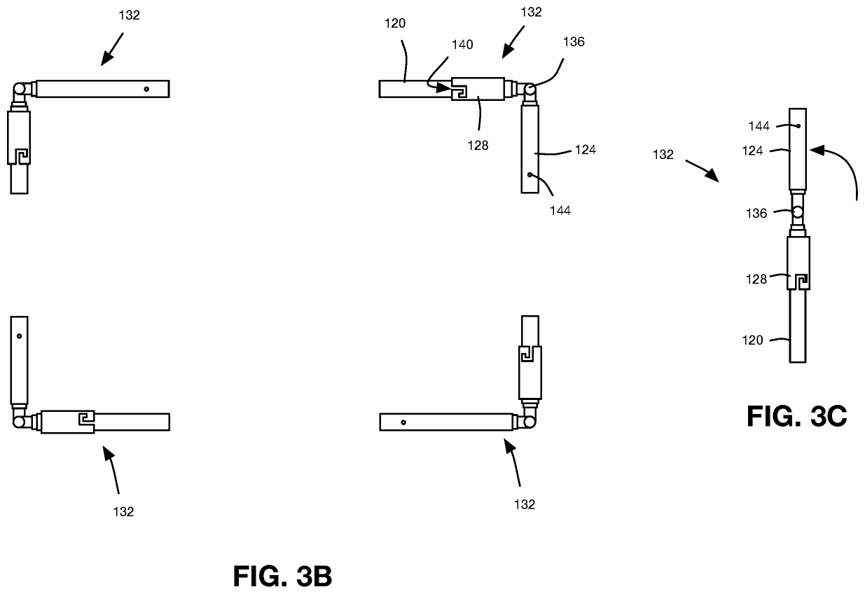

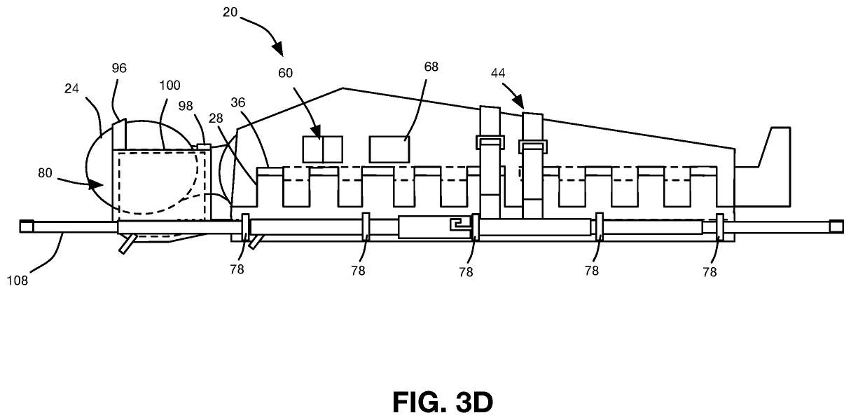

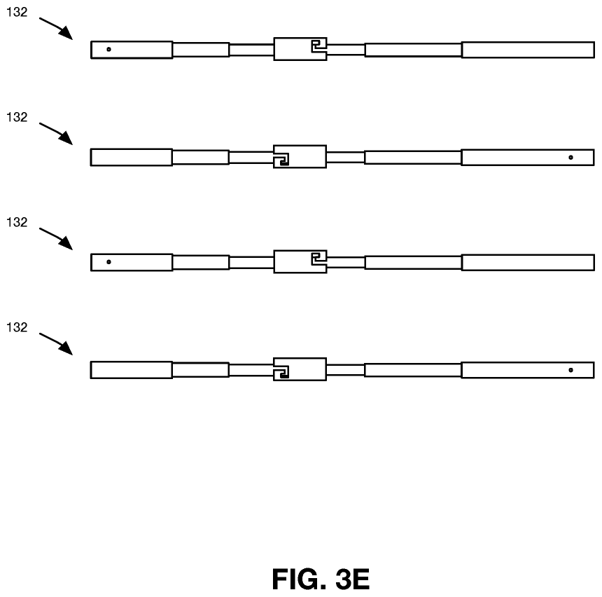

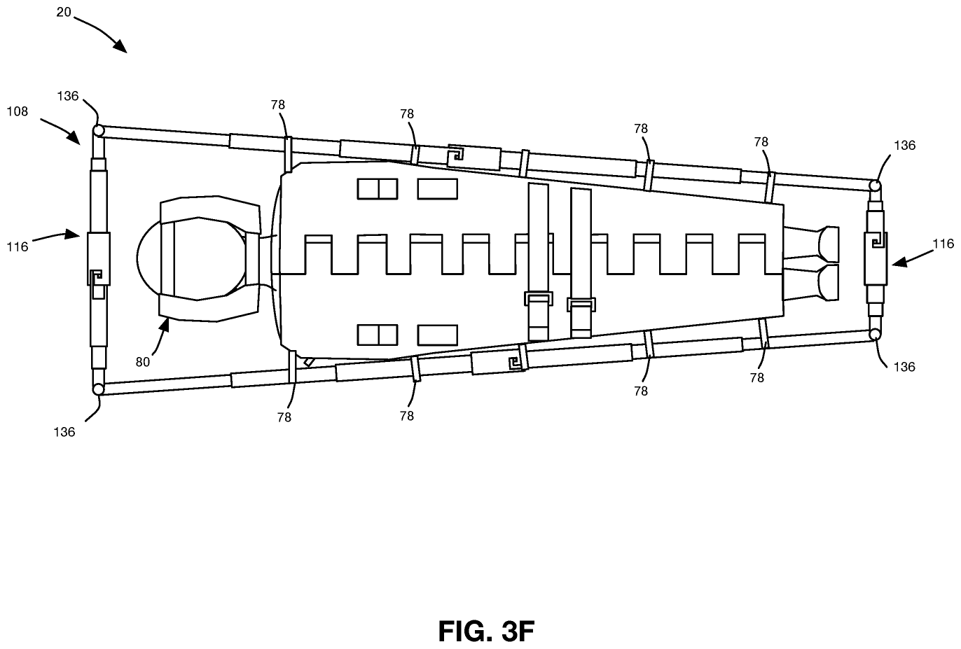

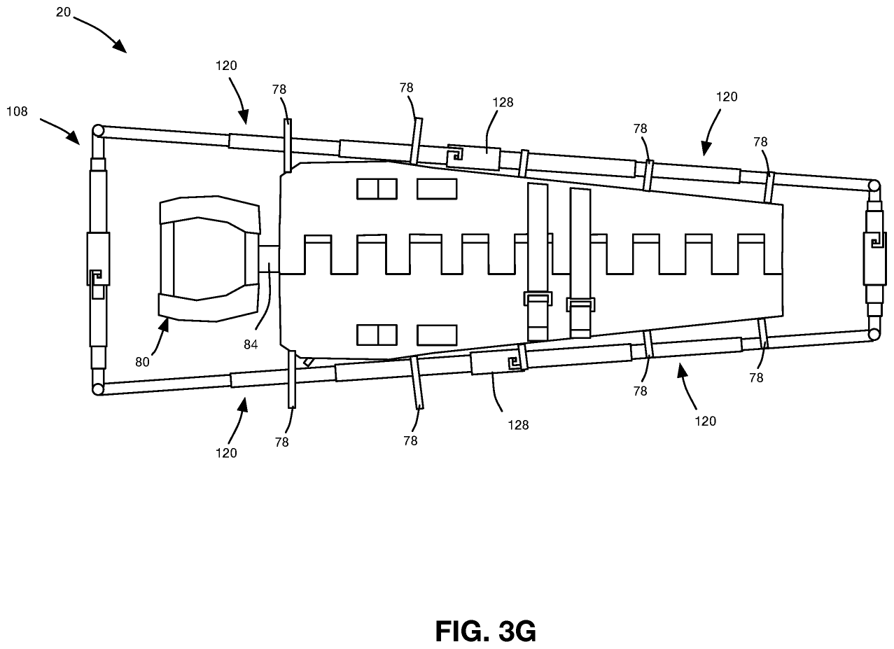

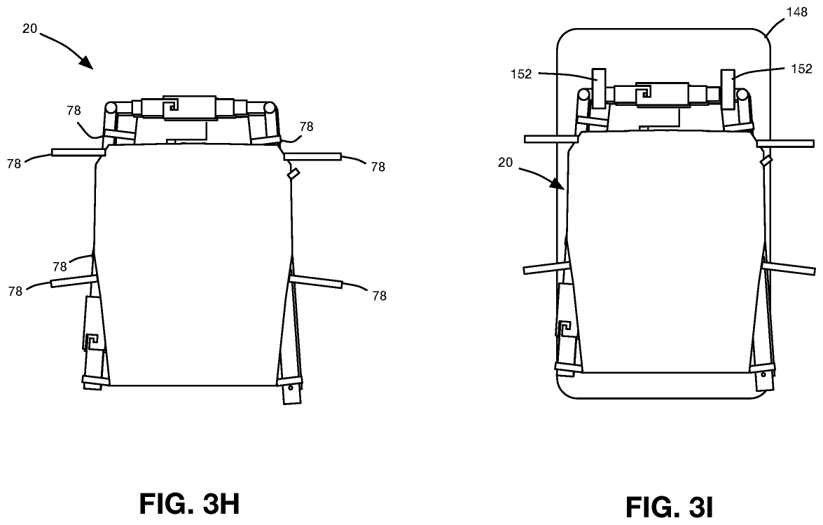

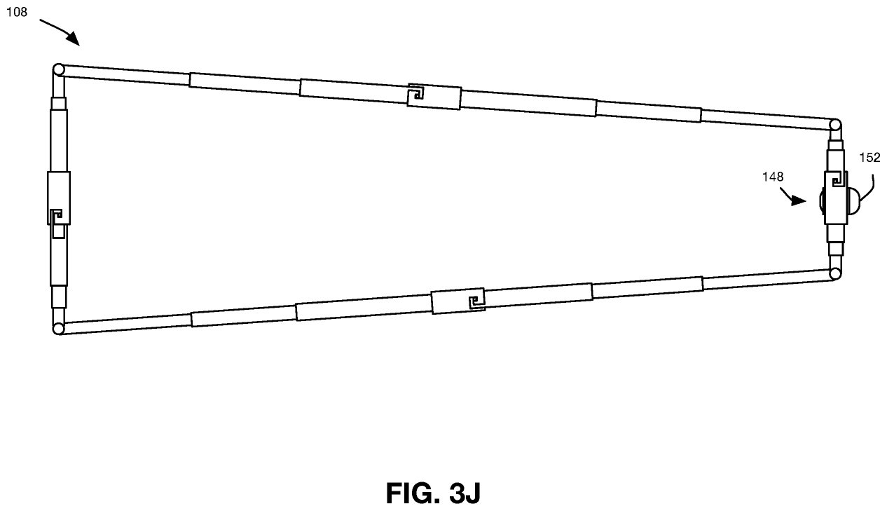

FIG. 3A shows a transportation structure 108 that is coupleable to the wrap 22 of FIGS. 1 and 2. The transportation structure 108 having two longitudinal sections 112 and two lateral sections 116 extending between the two longitudinal sections 112 towards distal ends thereof. Each of the longitudinal sections 112 include two telescoping longitudinal section portions 120, and each of the lateral sections 116 include two telescoping lateral section portions 124. Each of the telescoping longitudinal section portions 120 and the telescoping lateral section portions 124 can be locked to a particular length using any suitable means. Each pair of longitudinal section portions 120 is coupled together via locking sleeve 128 so that two longitudinal section portions 120 form a rigid structure. Similarly, each pair of lateral section portions 124 is coupled together via locking sleeve 128 so that two lateral section portions 124 form a rigid structure.

The transportation structure 108 can be collapsed by decoupling corresponding pairs of longitudinal section portions 120 and lateral section portions 124. This is done by sliding the locking sleeves 128 in a first direction, rotating it, and then sliding it in a second direction opposite the first direction. This process can be reversed to couple the parts together again.

FIG. 3B shows the transportation structure 108 of FIG. 3A being separated along each longitudinal section 112 and each lateral section 116 into four transportation structure segments 132 with the telescoping portions collapsed. Disassembly allows for the transportation structure 108 to be collapsed and made more compact so that it may be stored together with the deflated and folded or rolled up wrap 22 and head support 80. Each of the transportation structure segments 132 includes a longitudinal section portion 120 and a lateral section portion 124 that are pivotally connected at a pivot joint 136. The pivot joint 136 can be pivotally locked via any suitable means.

A hook-shaped slot 140 in the locking sleeve 128 positioned on one of the longitudinal section portion 120 and the lateral section portion 124 is dimensioned to receive and trap a protrusion 144 on the other of the longitudinal section portion 120 and the lateral section portion 124.

FIG. 3C shows one of the transportation structure segments 132 of FIG. 3B after pivoting of the lateral section portion 124 relative to the longitudinal section portion 120. In this manner, the transportation structure segment 132 can be collapsed to a different form to facilitate storage with the other three transportation structure segments 132.

FIG. 3D shows the transportation structure 108 having been assembled so that the longitudinal sections 112 pass through the loops 78 of the wrap 22. The lateral sections 116 and the ends of the longitudinal sections 112 may be grasped to carry the wrap 22 and person secured therein. As will be understood, the transportation structure 108 provides rigidity to the wrap 22 when connected to it in this manner.

FIG. 3E shows the four transportation structure segments of FIG. 3B after pivoting of the lateral section portions 124 relative to the longitudinal section portions 120 so that they are generally parallel, with the telescoping portions extended.

FIG. 3F shows the wrap of FIGS. 1 and 2 coupled to the transportation structure of FIG. 3A. The pivot joints 136 and the telescoping lateral portions 116 enable reconfiguration of the transportation structure 108 to more closely align with the general body shape of the person 24. Thus, differently shaped people can be accommodated by the transportation structure 108. Similarly, it is contemplated that different sized wraps can be provided to accommodate differently sized and shaped people.

Preferably, the wrap system folds down and is securable to the base of a large backpack (such as that of a military medic or a search-and-rescue person would have) for transportation prior to use as part of their kit.

FIG. 3G shows the wrap 22 and the transportation structure 108 after removal of the person. The head support 80 and the metal plate 84 can be decoupled from the wrap 22. Further, the longitudinal portion sections 120 can be decoupled by unsecuring the locking sleeves 128. The bladder of the wrap 22 can be deflated and the wrap 22 can be folded. In order to facilitate the collapsing of the wrap system 20, the wrap 22 may be decoupled from one or both of the portions of the transportation structure 108.

FIG. 3H shows the wrap 22 folded and the transportation structure 108 separated into two portions, with the wrap 22 still being coupled to one of the two portions of the transportation structure 108. The collapsed wrap system 20 can then be stored in or connected to a backpack or other carrying device.

FIG. 3I shows the wrap system 20 coupled to a backpack 148 via a set of straps 152. In this manner, the wrap system 20 can be readily carried by a field medic or the like.

FIG. 3J shows the transportation structure 108 of FIG. 3J with a wheel structure 148 secured thereto towards a foot end thereof. The wheel structure 148 includes a wheel 152 that may be inflatable to facilitate storage.

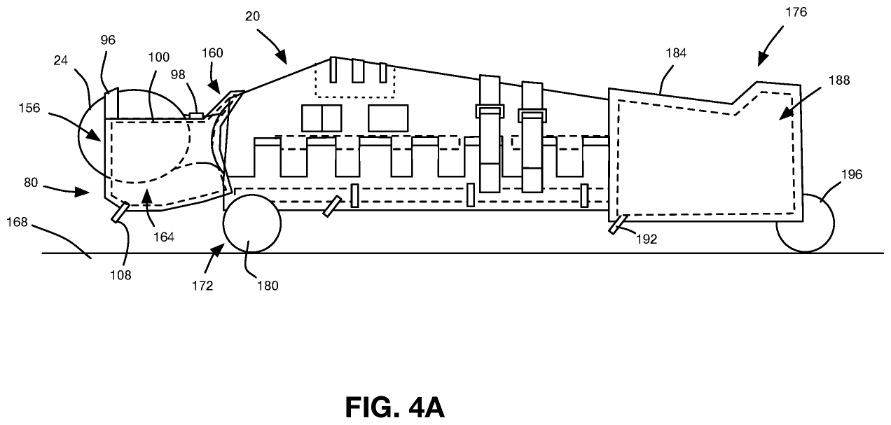

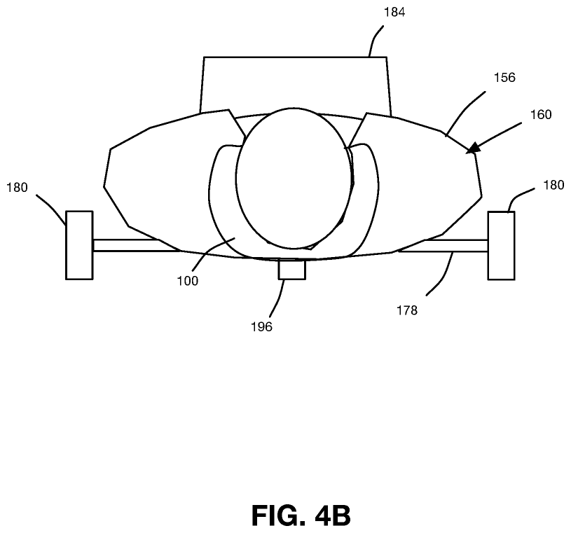

FIGS. 4A and 4B show the wrap 22 in use with a head support 156 in accordance with another embodiment. The head support 156 is similar to the head support 80 of FIGS. 1 and 2 and like elements are numbered accordingly. An attachment structure of the head support 156 includes a shoulder engagement portion 160 that is dimensioned to abut against the shoulders of the person 24. The shoulder engagement portion 160 can be coupled to the wrap 22 via any type of suitable means, such as hook-and-loop fabric patches, snaps, zippers, etc.

A bladder 164 is positioned within the exterior shell of the head support 156. The bladder 164 extends across a posterior side of the head and around the lateral sides of the head in the lateral support members 100. In addition, the bladder 164 extends into the shoulder engagement portion 160. It is fully enclosed within the exterior ballistic nylon shell of the head support 156, but can also be partially or fully externally positioned relative to the external ballistic nylon shell. A valve 108 that is reopenably sealable enables fluid communication with the bladder 164 to fill the bladder 164 with a fluid, and to drain the fluid from the bladder 164. In the illustrated and described embodiment, the fluid is air, but can be any other suitable fluid.

As the head support 156 is principally made of flexible materials, when the bladder 164 is generally empty of air, the head support 156 can be collapsed and folded or rolled to package the wrap 22 for carrying, such as in a backpack. When the bladder 164 is filled with air to a sufficient pressure, the bladder 164 becomes rigid. As a result, when the head of the person 24 is positioned within the head support 156 and the bladder 164 is filled, the head support 156 restricts movement of the head relative to the body of the person 24. The shoulder engagement portion 160 presses sufficiently firmly against the shoulders of the person 24 and, in effect, grasps the shoulders by extending both down the anterior and posterior sides of the shoulders. In conjunction with the connection(s) between the shoulder engagement portion 160 and the wrap 22, the inflation of the shoulder engagement portion 160 restricts movement of the head support 156 relative to the wrap 22.

As will be appreciated, the bladder 164 within the head support 156 provides cushioning to reduce the impact of bumping with objects in an environment.

The wrap 22 is also fitted with a rolling structure for rolling transportation of the person 24 over a surface 168. The rolling structure includes a torso wheel structure 172 and a boot wheel structure 176. The torso wheel structure 172 includes an axel 178 that can be secured to the wrap 22 via any suitable means or is built into the wrap 22, and a wheel 180 freely rotatably coupled to the axel at distal ends thereof.

The boot wheel structure 176 has a boot portion 184 made of a ballistic nylon shell with a bladder 188 therein. The bladder 188 can be filled or emptied via a valve 192. A wheel 196 (inflatable in one configuration) is rotatably coupled to the boot portion 184. The boot portion 176 can be fitted over the feet of the person in the wrap 22 and then inflated so that it becomes more rigid.

Using the illustrated structures, handles can be provided on the wrap 22 towards the shoulders to facilitate reorienting the person 24 upright and rolling the person 24 along on the 196 of the boot wheel structure 176.

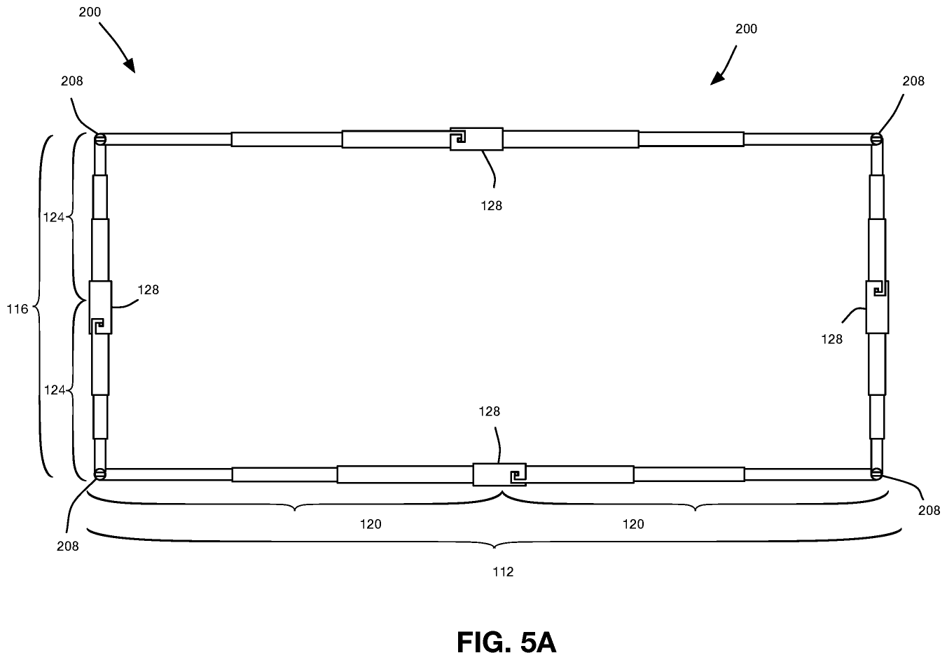

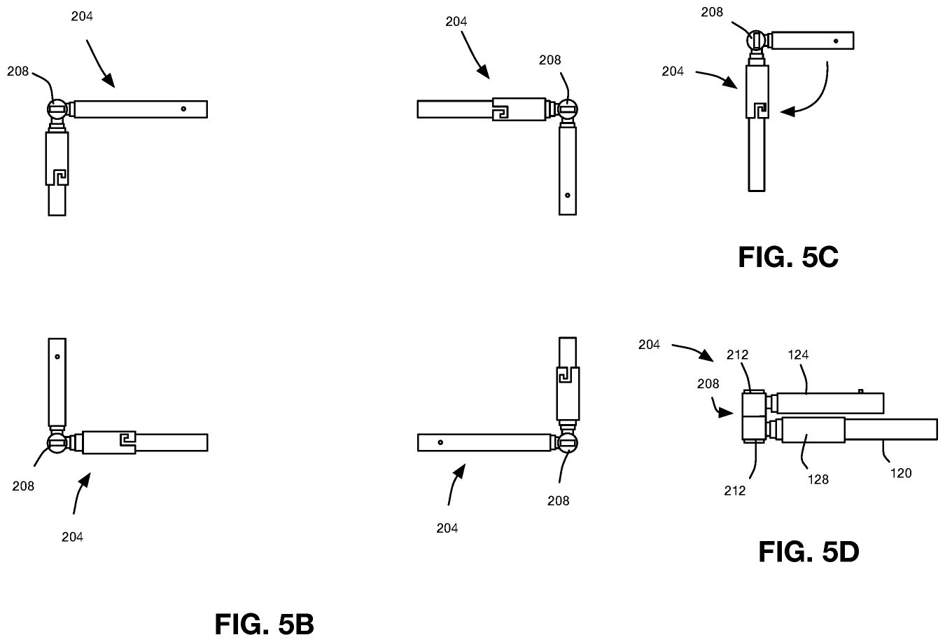

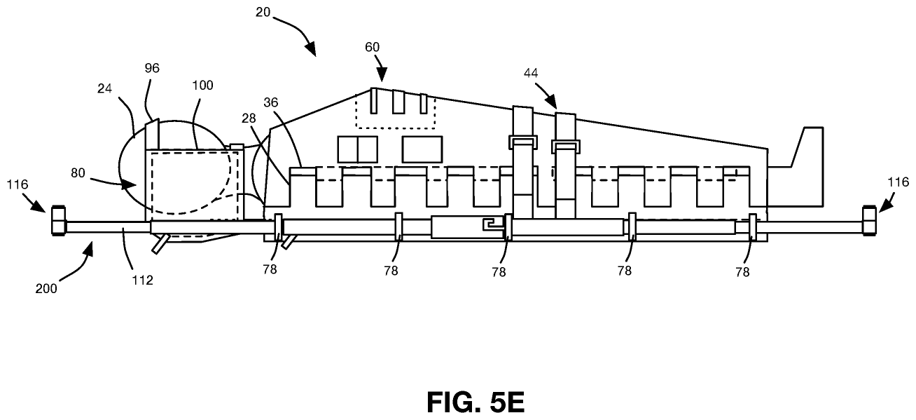

FIGS. 5A to 5D show a transportation structure 200 in accordance with another embodiment that is coupleable to the wraps of FIGS. 1, 2, and 4. Generally like elements to those of FIGS. 3A to 3C are numbered similarly. Each of four transportation structure segments 204 include a longitudinal section portion 120 that is pivotally connected to a lateral section portion 124 at a pivot joint 208. Each of the pivot joints 208 includes a pair of internal toothed interfaces that are pushed into contact with one another via springs but releasable by rotating controls 212.

FIG. 5D shows one of the transportation structure segments 204 after pivoting of the longitudinal section portion 120 relative to the lateral section portion 124. As a result, the compacted transportation structure segments 204 also have a generally smaller footprint.

FIG. 5E shows the transportation structure 200 having been assembled so that the longitudinal sections 112 pass through the loops 78 of the wrap 22. The lateral sections 116 are elevated relative to the longitudinal sections 112 and thus can be more readily grasped when the transportation structure 200 is resting on a surface, such as a floor or a ground.

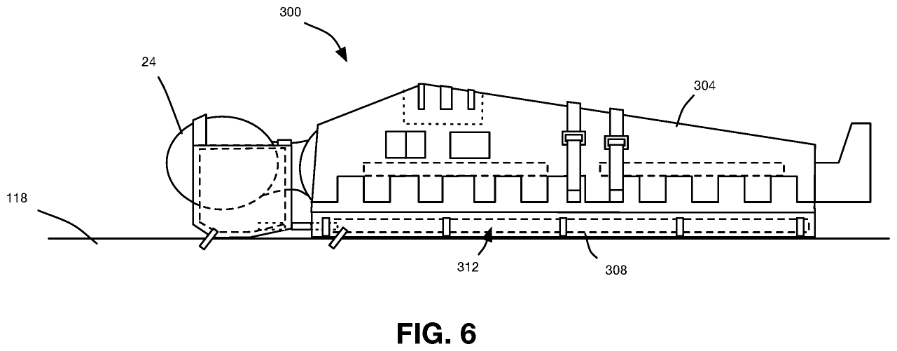

FIG. 6 shows a wrap system 300 in accordance with a further embodiment similar to the wrap system 20 of FIGS. 1 and 2, but having a wrap 304 that is separate from a fluid-filled support structure 308 secured to it. The support structure 308 has at least one bladder 312 fillable/filled with a fluid, such as air, that provides rigidity to the wrap 304. The support structure 308 can be secured to the wrap 304 via any means that connects the wrap 304 and the support structure 308 at at least various positions distributed over their area of contact. Examples can include hook-and-loop fabric patches, snaps, etc.

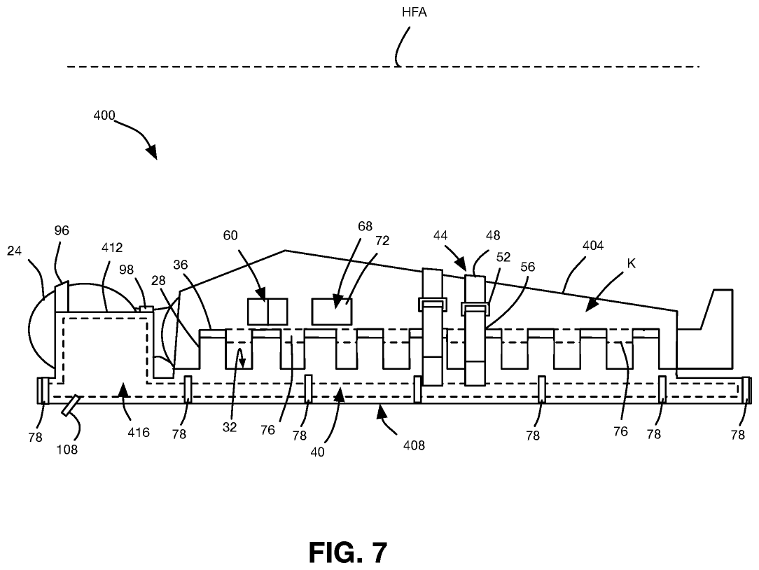

FIG. 7 shows a wrap system 400 in accordance with yet another embodiment, wherein a wrap 404 has a support portion 408 that extends from head to foot of the person 24. Elements similar to those of previous embodiments are numbered similarly and their description above applies as will be understood. The support portion 408 includes a pair of lateral head support members 412 that flank the head of the person 24 to restrict lateral movement and rotation of the head. A bladder 416 of the support portion 408 extends along the length of the wrap 404 from head to foot and into the lateral head support members 412. In other configurations, separate bladders can be provided, such as for the lateral head support members.

In other embodiments, the support structure can be separate from the wrap and securable to the wrap generally along the wrap adjacent to the posterior side of the person when the wrap is secured thereon.

Although specific advantages have been enumerated above, various embodiments may include some, none, or all of the enumerated advantages.

Persons skilled in the art will appreciate that there are yet more alternative implementations and modifications possible, and that the above examples are only illustrations of one or more implementations. The scope, therefore, is only to be limited by the claims appended hereto.

1. A wrap system for immobilizing a person, comprising a wrap dimensioned to be snugly securable around a person and extending from at least the upper torso to at least below knees of the person to restrict independent movement of limbs of the person, the wrap including a support structure including at least one fluid chamber positionable along a posterior side of the person when the wrap is secured around the person, the fluid chamber being reopenably sealable to enclose a fluid therein, the support structure immobilizing relative movement of a spine of the person.

2. The wrap system of claim 1, wherein the support structure is dimensioned to provide a head support structure for restricting movement of a head of the person relative to the torso of the person, the head support structure having a head restraint structure restricting movement of the head of the person relative to the head support structure.

3. The wrap system of claim 2, wherein the head restraint structure includes a strap extending across a forehead of the person.

4. The wrap system of claim 2, wherein the head restraint structure includes lateral support members extending along lateral sides of the head of the person to restrict rotation of the head.

5. The wrap system of claim 2, wherein the head restraint structure includes a head-supporting fluid chamber for cushioning the head of the person when positioned thereon.

6. The wrap system of claim 1, further comprising a head support structure coupleable to or extending from the wrap for restricting movement of a head of the person relative to the torso of the person, the head support structure having a head restraint structure restricting movement of the head of the person relative to the head support structure.

7. The wrap system of claim 6, wherein the head restraint structure includes a strap extending across a forehead of the person.

8. The wrap system of claim 6, wherein the head restraint structure includes lateral support members extending along lateral sides of the head of the person to restrict rotation of the head.

9. The wrap system of claim 6, wherein the head restraint structure includes a head-supporting fluid chamber for cushioning the head of the person when positioned thereon.

10. The wrap system of claim 1, wherein the wrap includes hip straps to further restrict movement of a pelvis of the person.

11. The wrap system of claim 1, wherein the wrap includes a chest port to facilitate access to a chest of the person when the wrap is secured around the person.

12. The wrap system of claim 1, wherein the wrap includes a vein port to facilitate access to an antecubital fossa of the person when the wrap is secured around the person.

13. The wrap system of claim 1, wherein the support structure includes at least a second fluid chamber that is positionable along the posterior side of the person when the wrap is secured around the person, the at least a second fluid chamber being reopenably sealable to enclose a fluid therein.

14. The wrap system of claim 1, wherein the wrap includes at least two sleeves extending generally along a head-to-foot axis of the person for receiving stiffener members.

15. The wrap system of claim 1, further comprising a transportation structure that is coupleable to the wrap to provide rigidity to the wrap when the wrap secured around the person is being moved.

16. The wrap system of claim 15, wherein the transportation structure includes two longitudinal sections extending along the wrap generally along a head-to-foot axis of the person when the wrap is secured around the person.

17. The wrap system of claim 16, wherein the transportation structure includes two lateral sections extending between the two longitudinal sections towards distal ends thereof.

18. The wrap system of claim 17, wherein each longitudinal section is formed from two longitudinal section portions that are securable to one another, and each lateral section is formed from two lateral section portions that are securable to one another.

19. The wrap system of claim 18, wherein each longitudinal section portion is coupleable to a corresponding one of the lateral section portions.

20. The wrap system of claim 19, wherein each longitudinal section portion is pivotally coupleable to a corresponding one of the lateral section portions.

21. The wrap system of claim 1, further comprising a foot wheel structure that is coupleable to or forms part of the wrap towards feet of the person when the wrap is secured around the person, the foot wheel structure comprising a wheel for facilitating rolling travel over a surface.

22. The wrap system of claim 21, wherein the foot wheel structure includes an inflatable boot structure that is dimensioned to be positioned over at least the feet of the person.

23. The wrap system of claim 1, further comprising a torso wheel structure coupleable to or forming part of the wrap towards shoulders of the person when the wrap is secured around the person, the torso wheel structure including at least two wheels for facilitating rolling travel over a surface.
